(19) United States
(12) Patent Application Publication
(10) Pub. No.: US 2021/0338228 A1
(43) Pub. Date: Nov. 4, 2021

(54) SURGICAL LINE FIXATION DEVICE AND METHODS OF USE THEREOF

(71) Applicant: The Board of Supervisors of Louisiana State Universityand Agricultual amd Mechanical Colleg, Baton Rouge, LA (US)

(72) Inventors: Mandi Jean LOPEZ, St. Gabriel, LA (US); Gerhard Salomo MUNDINGER, New Orleans, LA (US)

(21) Appl. No.: 17/280,085

(22) PCT Filed: Sep. 25, 2019
(86) PCT No.: PCT/US2019/052890

§ 371 Date: Mar. 25, 2021

Related U.S. Application Data
(60) Provisional application No. 62/736,212, filed on Sep. 25, 2018.

(51) Int. Cl. A61B 17/04; A61F 2/08

(52) U.S. Cl. CPC A61B 17/0401; A61F 2/0811; A61B 2017/0409; A61B 17/1675; A61B 2017/045; A61F 2002/0829; A61F 2210/0004; A61B 2017/044

(57) ABSTRACT

This invention is directed surgical line fixation devices and kits, and methods for using the same.

This application claims priority from U.S. Provisional Application No. 62/736,212 filed on Sep. 25, 2018, the entire contents of which is incorporated by reference herein.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed surgical line fixation devices and kits, and methods for using the same.

### BACKGROUND OF THE INVENTION

The development of suture anchoring devices has played an important role in the transition of from open to arthroscopic surgical procedures. However, there have been reported complications during use of such anchors, including loosening, migration, and chondral injury. See, for example, Dhawan, Aman, et al. “Complications of bioabsorbable suture anchors in the shoulder.” The American journal of sports medicine 40.6 (2012): 1424-1430.

### SUMMARY OF THE INVENTION

A surgical line fixation device is disclosed herein. In one embodiment the device comprises an anchor configured to at least partially reside within a rigid structure, the anchor being further configured to hold one or more sutures; and a cap configured to secure the one or more sutures within the anchor.

In an embodiment, the anchor comprises a top portion and a bottom portion, wherein the top portion is configured to hold one or more sutures and is configured to receive the cap, and wherein the bottom portion is configured to permit attachment of the anchor to the rigid structure.

In an embodiment, the anchor comprises an interior space with one or more crossbars disposed therein. The crossbars can be configured to receive and hold at least a portion of one or more sutures.

In an embodiment, the anchor comprises one or more sections configured to receive and hold at least a portion of the suture. The sections can be displaced on the top portion of the anchor. In embodiments, the one or more sections comprise wedges, notches, grooves, gaps, channels, or indentions. In embodiments, the sections can also function as locking mechanisms, which can be complementary to those on the cap. For example, the top portion of the anchor can comprise one or more locking mechanisms, wherein the one or more locking mechanisms comprise a structure that is complementary to one or more locking mechanisms on the cap.

Likewise, the cap can comprise one or more locking mechanisms that comprise a structure that is complementary to one or more locking mechanisms on the anchor. Such locking mechanisms can hold the cap in place when affixed to the anchor.

Furthermore, both the cap and the anchor can comprise one or more complementary securing mechanisms. For example, the cap can comprise one or more securing mechanisms comprising a structure that is complementary to one or more securing mechanisms on the anchor. In embodiments, the securing mechanism comprises a flange or threads. The securing mechanisms can be displace on the top portion of the anchor.

In embodiments, the anchor can further comprise a structure configured to permit attachment of the anchor to the rigid structure. For example, the structure can be a threaded portion or a flange configured to permit attachment of the anchor to the rigid structure.

Aspects of the invention can further comprise an applicator. In embodiments, the applicator can comprise a dispenser for positioning the anchor, the cap, or both. For example, the dispenser can comprise a first alignment mechanism, wherein the first alignment mechanism is configured to interact with a second alignment mechanism disposed upon the cap, the top of the anchor, or both. In embodiments, the first and second alignment mechanisms are complementary to one another. In embodiments, the second alignment mechanism is configured to receive the first alignment mechanism. In embodiments, the first alignment mechanism is configured to receive the second alignment mechanism. In embodiments, the second alignment mechanism is removable.

In embodiments, the applicator further comprises one or more aligning bars and the anchor further comprises one or more aligning notches configured to receive the aligning bars. For example, the aligning notches can be dispersed on the top portion of the anchor.

In embodiments, the anchor further comprises one or more aligning bars and the applicator further comprises one or more aligning notches configured to receive the aligning bars.

In embodiments, the anchor is to be affixed to a rigid structure, such as a hard tissue of a subject. In embodiments, the rigid structure comprises cartilaginous tissue, a bone, or a tooth of the subject. In embodiments, the rigid structure comprises cortical bone, cancellous bone, flat bones, long bones, enamel, cartilage, or fibrocartilage.

In embodiments, the device is substantially cylindrical.

In embodiments, the device is polygonal.

In embodiments, the anchor is configured to be removable when least partially residing within the rigid structure.

In embodiments, the anchor is configured to reversibly hold the one or more sutures.

In embodiments, the anchor is configured to be permanent, semi-permanent, or removable.

In embodiments, the device is configured to hold up to 20 sutures, up to 10 sutures, or up to 5 sutures.

In embodiments, the device is configured to permit adjustment of a tension within each of the one or more sutures.

In embodiments, the interior space defines an interior surface of the anchor, and the interior surface comprises one or more flanges configured to secure the cap to the anchor. Such flanges can be referred to as a securing mechanism.

In embodiments, the interior space defines an interior surface, the interior surface comprises a first plurality of threads; the cap comprises a second plurality of threads; and the first plurality of threads and the second plurality of threads are configured to engage one another. Such threads can be referred to as a securing mechanism.

In embodiments, the device comprises a metal or a synthetic polymer.

In embodiments, the device comprises a bioprinted material, such as natural or synthetic protein, a mineral, or a combination thereof. Non-limiting examples of protein comprises collagen, laminin, or a combination thereof. Non-limiting examples of the mineral comprises tricalcium phosphate.

In embodiments, the device is configured for use in anterior cruciate ligament reconstruction or repair, rotator cuff repair, plastic or reconstructive face or head soft tissue surgery, elbow or ankle tendon and ligament repair or stabilization, cleft palate repair, temporal mandibular joint ligaments or articular disc repair, dental applications, gum stabilization, upper airway surgery, facial muscle anchoring, facial paralysis reconstruction, cosmetic facial surgical procedures (including browlift and canthoplasty), rhytidectomy, platysmaplasty, hand tendon repair, belt lipectomy, thigh lifting, upper extremity tendon and muscle reconstruction, temporalis muscle suspension, medial canthal reconstruction, facial soft tissue suspension or combinations thereof. For example, the upper airway surgery comprises larynx repair, pharynx repair, or a combination thereof.

Aspects of the invention are also drawn towards a method of securing soft tissue to a rigid structure. For example, the method comprises drilling a channel into the rigid structure; inserting an anchor described herein into the rigid structure; suturing a soft tissue to the anchor with the one or more sutures; modifying the tension of the one or more sutures; and placing the cap on the anchor to secure the sutures in place. In embodiments, the cap is secured to the anchor with a securing mechanism, such as threads or a flange. In embodiments, the cap is placed on the anchor with an applicator.

The method can further comprise looping each of the one or more sutures around one or more crossbars disposed within an interior space of the anchor; passing the one or more sutures through the soft tissue; temporarily securing at least a portion of the one or more sutures within a section; tensioning the one or more sutures; and affixing the cap to the anchor. In embodiments, the sutures can exit the anchor through the sections in the anchor, such as the wedge-shaped sections.

In embodiments, the cap further comprises one or more locking mechanisms, and embodiments can further comprise aligning the one or more locking mechanisms with the one or more locking mechanisms of on the anchor before affixing the cap to the anchor.

Embodiments can further comprise the steps of aligning the first alignment mechanism with the second alignment mechanism; and using the applicator to affix the cap to the anchor.

Aspects of the invention are further drawn to a kit for securing soft tissue to a rigid structure comprising embodiments of the device as described herein and instructions for use.

In embodiments, the kit can further comprise a drill.

In embodiments, the kit can comprise an anchor comprising a first threaded portion configured to permit reversible attachment of the anchor to the rigid structure; and the kit further comprises a second anchor comprising a second threaded portion that is larger than the first threaded portion.

Embodiments of the kit can further comprise a plurality of differentially sized anchors.

Aspects of the invention are also drawn to a kit for securing soft tissue to a rigid structure. For example, the kit comprises a drill; a plurality of differentially sized anchors, each anchor being configured to at least partially reside within a rigid structure, each anchor being further configured to hold one or more sutures; a cap configured to secure the one or more sutures within the anchors; an applicator comprising a dispenser and a first alignment mechanism, wherein the first alignment mechanism is configured to interact with a second alignment mechanism disposed upon the cap; or a combination thereof, and instructions for use.

Aspects of the invention are drawn towards a surgical fixation device. In embodiments, the device comprises an anchor configured to at least partially reside within a rigid structure, the anchor being further configured to hold one or more soft tissue sections; and a cap configured to secure the one or soft tissue sections within the anchor. In embodiments, the anchor further comprises one or more sections configured to receive and hold at least a portion of the tissue sections; and the cap comprises one or more locking mechanisms, wherein the locking mechanisms each comprise a structure that is complementary to a locking mechanism on the anchor.

Embodiments can further comprise an applicator comprising a dispenser and a first alignment mechanism, wherein the first alignment mechanism is configured to interact with a second alignment mechanism disposed upon the cap.

Further, aspects of the invention are drawn to a method of securing soft tissue to a rigid structure. For example, the method comprises drilling a channel into the rigid structure; inserting the anchor into the rigid structure; inserting one or more soft tissue sections into at least a portion of the anchor; modifying the tension of the one or more soft tissue sections; and placing the cap on the anchor to secure the one or more soft tissue sections in place.

The method can further comprise temporarily securing at least a portion of the one or more soft tissue sections within a notch, groove, gap, channel, or indentation; tensioning the one or more soft tissue sections; and affixing the cap to the anchor.

Other objects and advantages of this invention will become readily apparent from the ensuing description.

### BRIEF DESCRIPTION OF THE FIGURES

FIG. 1 shows a side perspective view of an exemplary anchor of the suture anchoring device.

FIG. 2 provides a top perspective view of an exemplary anchor of the suture anchoring device.

FIG. 3 shows a side view of an exemplary cap and aligning cube of the suture anchoring device.

FIG. 4 shows a perspective view of an exemplary cap and aligning cube of the suture anchoring device.

FIG. 5 shows a perspective view of an exemplary applicator of the suture anchoring device.

FIG. 6A shows a side schematic view of the surgical line fixation system, under an additional aspect.

FIG. 6B provides a cross sectional view of the surgical line fixation system of FIG. 6A.

FIG. 7A shows a top perspective view of the surgical line fixation system of FIG. 6A.

FIG. 7B shows a bottom perspective view of the surgical line fixation system of FIG. 6A. In this view, the external housing of the applicator is shown in phantom to reveal the dispenser therein.

FIG. 8 provides a side perspective view of the FIG. 6A applicator with the dispenser exploded above the external housing of the applicator.

FIG. 9A provides a bottom perspective view of the FIG. 6A cap.

FIG. 9B provides a top perspective view of the FIG. 6A cap.

FIG. 10A shows a perspective view of the anchor of FIG. 6A.

FIG. 10B provides a top perspective view of the anchor of FIG. 6A.

FIG. 11A shows the cap of FIG. 6A moving in the direction of the arrow to be loaded onto the applicator of FIG. 6A.

FIG. 11B shows the applicator/cap complex moving toward the anchor in the direction of the arrow.

FIG. 11C shows the applicator complex aligned over the anchor.

FIG. 11D shows the cap of the applicator/cap complex being inserted into or upon the anchor through depression of the dispenser handle in the direction of the arrow.

FIG. 11E shows the cap fully inserted into or upon the anchor.

FIG. 12 shows an embodiment of the anchor.

FIG. 13 shows an embodiment of the applicator cylinder.

FIG. 14 shows an embodiment of the applicator plunger.

FIG. 15 shows an embodiment of the anchor.

FIG. 16 shows an embodiment of the system.

FIG. 17 shows an embodiment of the system.

FIG. 18 shows an embodiment of the anchor.

FIG. 19 shows an embodiment of the cap.

FIG. 20 shows an embodiment of the applicator cylinder.

FIG. 21 shows an embodiment of the applicator plunger.

FIG. 22 shows an embodiment of the system.

FIG. 23 shows an embodiment of the anchor.

FIG. 24 shows an embodiment of the cap.

FIG. 25 shows an embodiment of the applicator cylinder.

FIG. 26 shows an embodiment of the applicator plunger.

FIG. 27 shows an embodiment of the system

FIG. 28 shows an embodiment of the anchor.

FIG. 29 shows an embodiment of the cap.

FIG. 30 shows an embodiment of the applicator cylinder.

FIG. 31 shows an embodiment of the applicator plunger.

FIG. 32 shows a bottom up view of an embodiment of the invention.

FIG. 33 shows a forward facing view of an embodiment of the invention.

FIG. 34 shows a perspective of an embodiment of the invention.

FIG. 35 shows a side profile view of an embodiment of the invention.

FIG. 36 shows a top down view of an embodiment of the invention.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

Current suture anchoring devices utilize deployable “anchoring” devices in which the anchor is physically deployed after the device is bored into cartilaginous tissues or bone. Anchors cannot be removed and the fixation is consequently irreversible. Additionally, current suture anchors fix the sutures such that the sutures cannot be removed as they are attached to the core components of the device. Typically, there is only one suture per device. In the rare device that has multiple removable sutures, there is a single anchor point in the device. Because knots are required to secure the tissue, the tension cannot be adjusted nor distributed among the sutures. Knots also introduce weak points into the suture, which can result in breaking of the suture and dissociation of the soft tissue from the anchor or fixation point. An additional disadvantage of knotted suture systems is that such systems require additional time and complicate the suturing process. Another drawback to existing devices is that they require a significant tunnel for application and engage primarily cancellous bone as opposed to stronger cortical bone for fixation. Finally, for the few devices that do permit removable sutures, there is a single anchor point in the device, which can result in clinically significant pull-out forces on the anchor. There have been reported complications during use of current suture anchors and suture anchor devices, including loosening, migration, and chondral injury.

Suture Anchoring System

Aspects of the present invention address these unmet needs, for example, providing for an improved suture anchoring device. For example, improvements of the suture anchoring device described herein include multiple sutures per anchor; ability to adjust tension of sutures on anchor; and ability to secure soft tissue to bone.

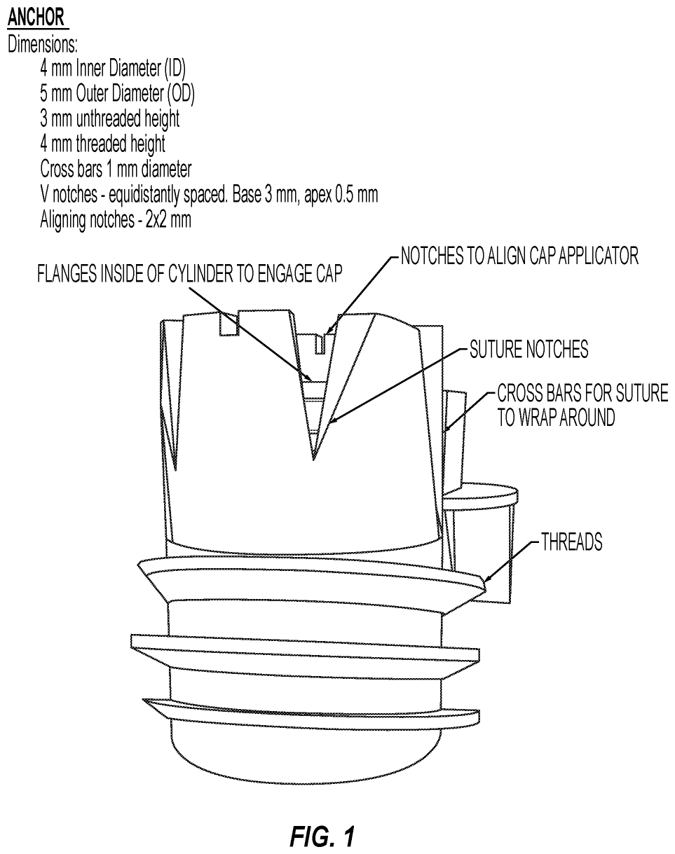

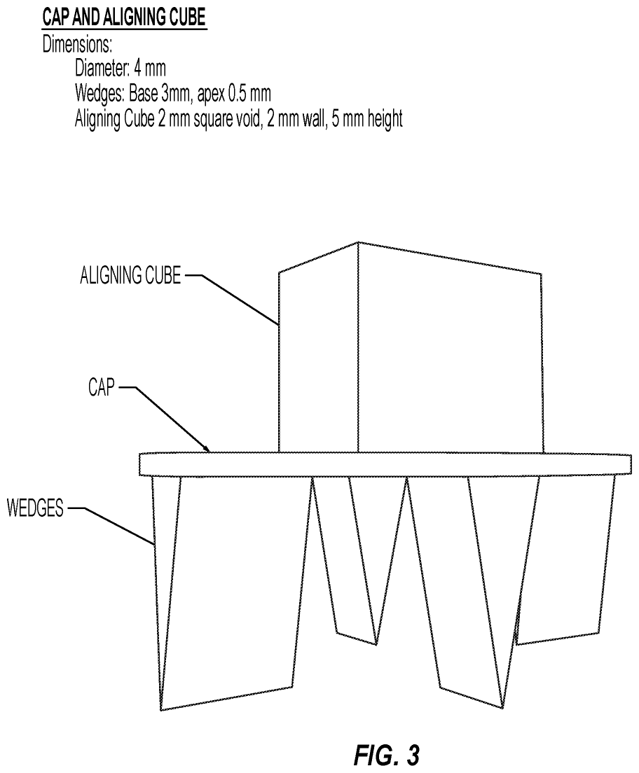

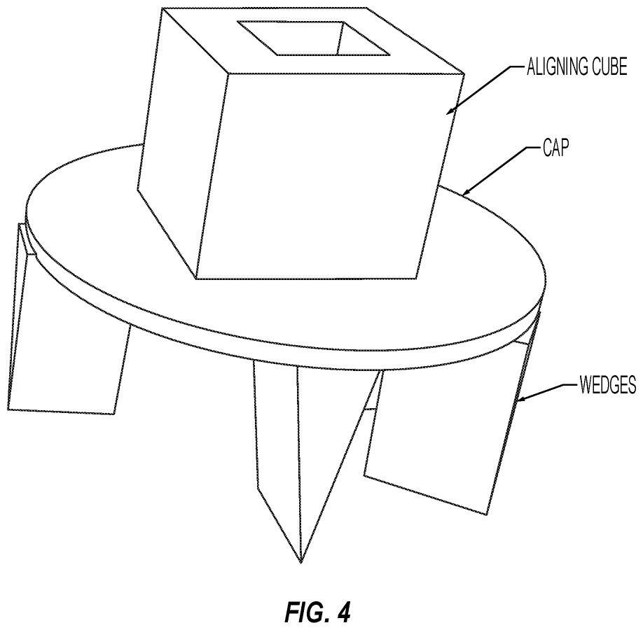

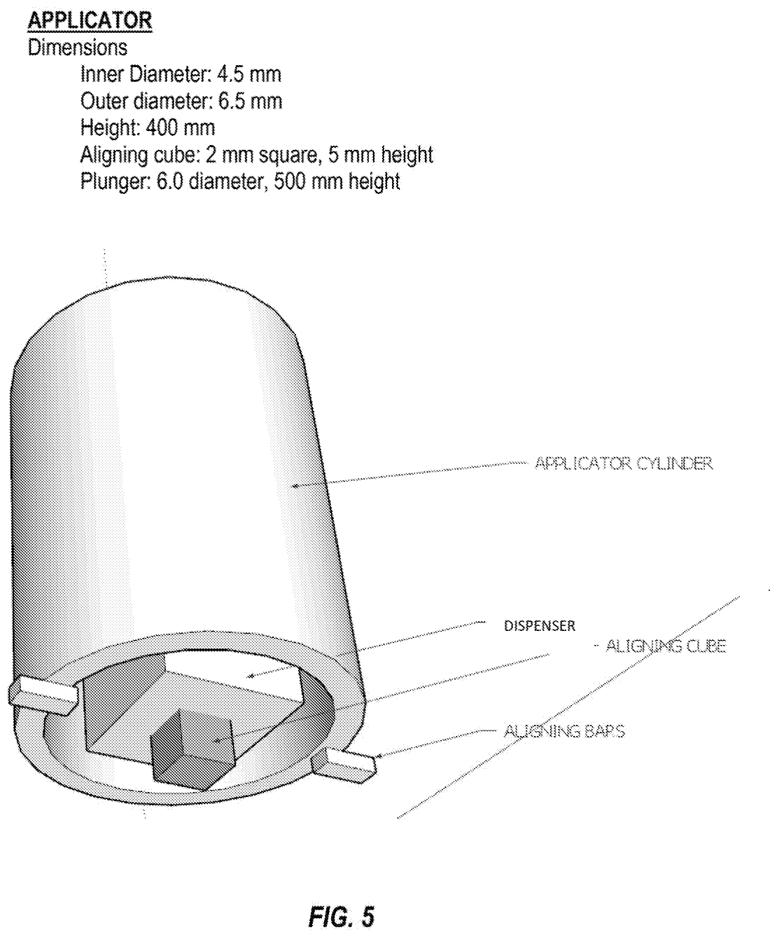

Referring to FIGS. 1-5, for example, a suture anchoring system in accordance with the invention is shown. The suture anchoring system can include a suture anchor (FIGS. 1 & 2), a cap (FIGS. 3 & 4), optionally comprising an aligning cube (FIG. 4), an applicator (FIG. 5), or any combination thereof. Such components can be provided as a kit.

FIG. 1 provides a side perspective view of an anchor under one embodiment. The FIG. 1 anchor comprises sections (such as wedge shaped sections) at the top of the anchor (i.e., top portion of the anchor), each section having a notch, groove, channel, indentation, or combination thereof configured to receive one or more sutures. In embodiments, the one or more sutures can be preloaded for convenience. Alternative embodiments can comprise sections of any shape, design, heights, widths, or configuration, so long as the section is configured to receive one or more sutures. For example, the height of the grooves can be altered so as to make the cap closer to or farther from the threads. In certain embodiments, the one or more notches, grooves, channels, indentations, or combinations thereof are configured to hold a portion of a tissue rather than a suture.

The skilled artisan will recognize that the sections can be configured to receive any thread type. In embodiments, the thread types can be varied depending on the location, purpose, and patient. That is, similar to cortical and cancellous bone screws. Thus, the anchor is configured to receive any thread parameter, pitch, depth, width, self-tapping.

Embodiments can further comprise a plurality of threads on the bottom portion of the anchor that permits the anchor to be reversibly attached to a rigid structure such as a hard tissue of a subject. The threads can be designed to be attached to a rigid structure by turning or screwing the anchor in clockwise motion, or can be designed to be attached to a rigid structure by turning or screwing the anchor in a counter clockwise motion. In other embodiments, anchor can be threadless. For example, the anchor can be held into place by the sutures themselves depending on the direction of the tensile force on the sutures. In still other embodiments, the anchor can be held into place by flanges, such as for those anchors that are to be permanent and thus very difficult to remove.

In embodiments, the anchor can be a permanent anchor. For example, a permanent anchor can be used for rotator cuff or other tendon and/or joint capsule repair.

In embodiments, the anchor can be a removable anchor. For example, a removable (or temporary) anchor can be used in the case of revision surgery or for incremental/staged surgeries characteristic of complex reconstruction. For example, the removable anchor can comprise threads that, when ready to be removed, will allow the anchor to be unscrewed from the implant site.

In embodiments, the anchor and/or cap can be absorbable, such as an anchor manufactured from bioabsorbable material. In many pediatric and adult plastic surgery applications for soft tissue suspension (such as forehead lifting, temporalis muscle resuspension, facelifting) the anchor has done it's work after a year when scarring and surgical field manipulation is completely healed. Once the healing is done, we want the anchor and cap to disappear (such as reabsorbed or degrade). For example, polylactic/polyglycolic acid can be used to make resorbable anchor and cap. The terms “bioabsorbable” or “biodegradable” can be used interchangeably.

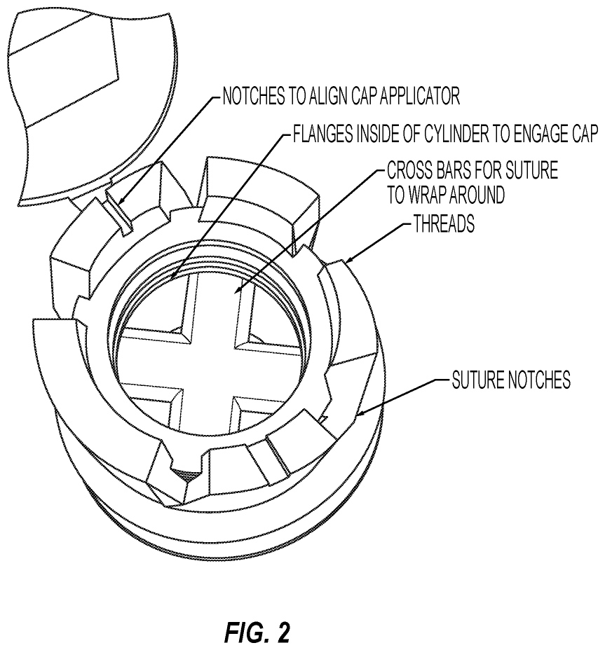

FIG. 2 provides a top perspective view of the anchor displayed in FIG. 1. This embodiment is hollow, comprising an interior space and an interior surface. Two crossbars are shown traversing the interior space of the anchor. Embodiments can comprise one, two, three, four, or five crossbars. The cross-bars, for example, are designed to receive sutures, which then exit the anchor through the sections, shown herein as wedge sections. As shown in FIG. 2, the top surface of the anchor can comprise one or more aligning notches configured to receive the aligning notches (i.e., aligning bars) of the applicator (discussed in more detail herein). In this embodiment, a plurality of flange can be seen along the interior surface of the anchor. The flanges can be configured to frictionally engage and hold the cap.

FIG. 3 shows a side view of a cap and, optionally, an aligning cube, under one embodiment. In embodiments, the cap is configured to be affixed to the top of the anchor and cover the anchor and secure sutures therein. In embodiments, the flanges on the cap interdigitate with the flanges on the inside of the anchor to affix the cap to the anchor. In embodiments, the cap can be low-profile to reduce palpability and visibility under the skin. Embodiments that comprise a cap are useful for securing small diameter sutures, and can be particularly suited for facial and hand surgical applications. Facial, hand, plastic, and periocular surgeries, for example, typically involve very small sutures and thin, friable tissues that cannot withstand the damage from passage of large sutures nor tension on the sutures themselves. Thus, very small sutures and devices are typically advantageous. The cap can comprise one or more locking mechanisms that are configured to fit within the sections of the anchor. The locking mechanism can comprise any shape, size, design, or configuration.

In embodiments, the cap can be a removably affixed to the anchor. A removable cap can be useful if, for example, the sutures need to be adjusted at any point in time. In other embodiments, the cap can be permanently affixed to the anchor, or can be semi-permanently affixed to the anchor. A semi-permanent cap can, for example, be removed as desired, but with difficulty.

In embodiments, the cap can further comprise features (such as square teeth) that allow the cap and anchor to be further screwed into the tissue, such as bone, after placement.

The embodiment displayed in FIG. 3 also shows a cube-shaped structure (i.e., aligning cube) that contains an alignment mechanism configured to interact with a complementary alignment mechanism on the applicator (discussed in more detail herein). In certain embodiments, the alignment mechanism (i.e., aligning cube) can removable to permit the cap to be flush with top of the anchor after being affixed thereto. Certain embodiments with a removable alignment mechanism can comprise a perforation or alternate means for removing the alignment mechanism.

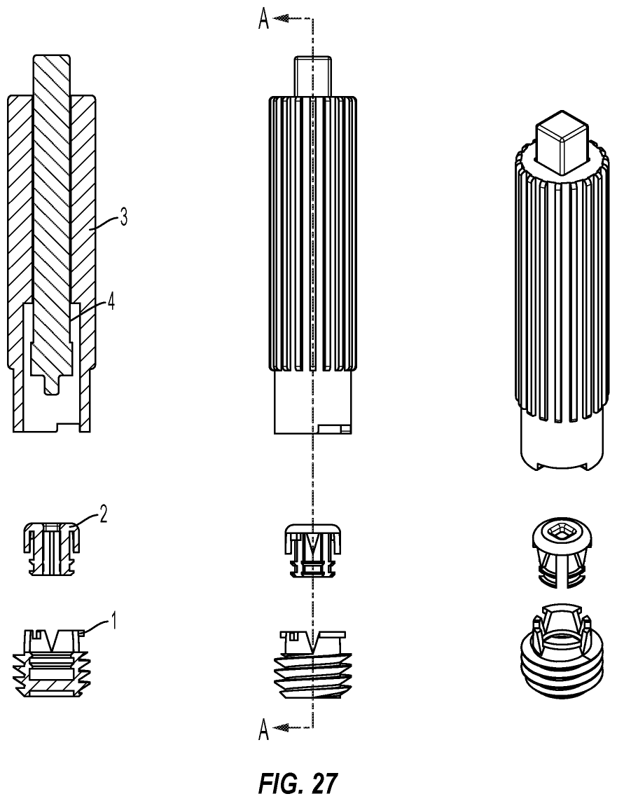

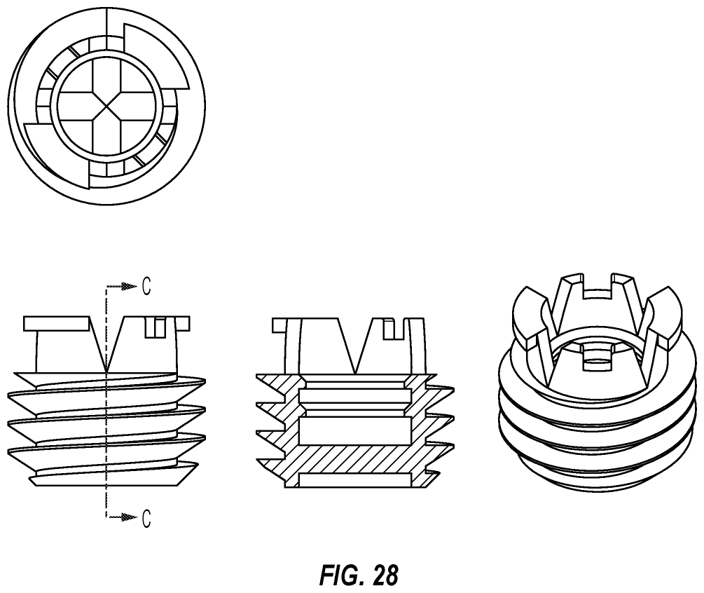

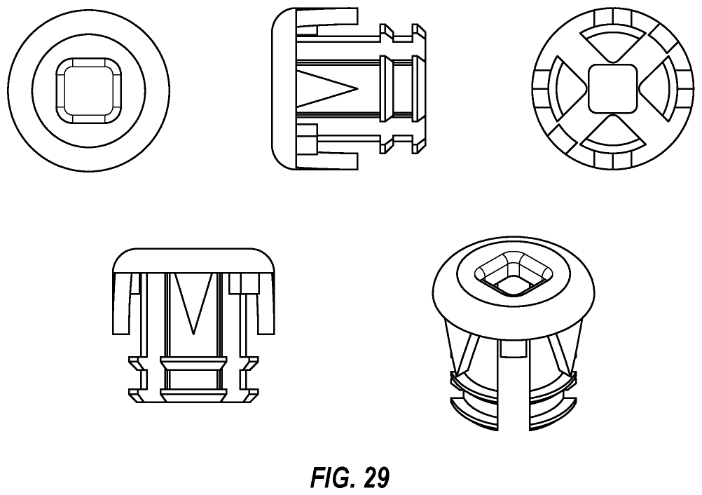

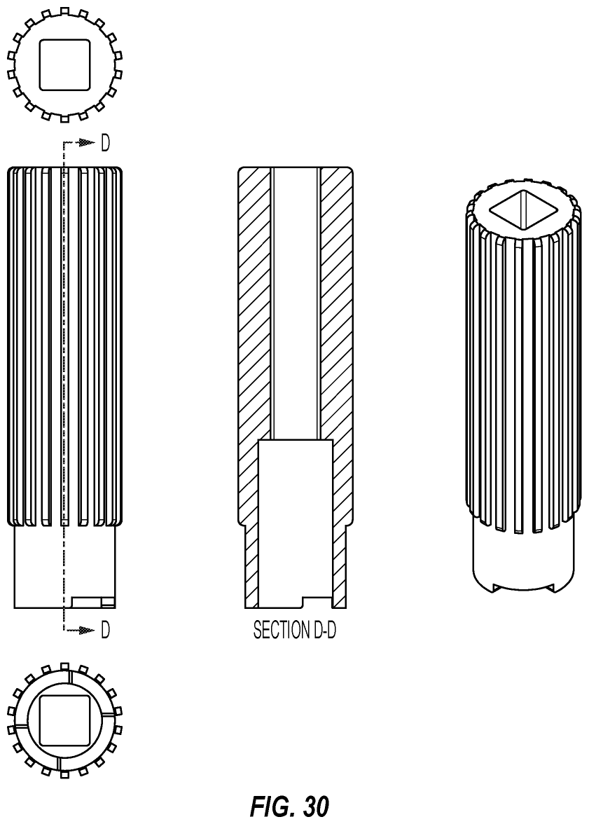

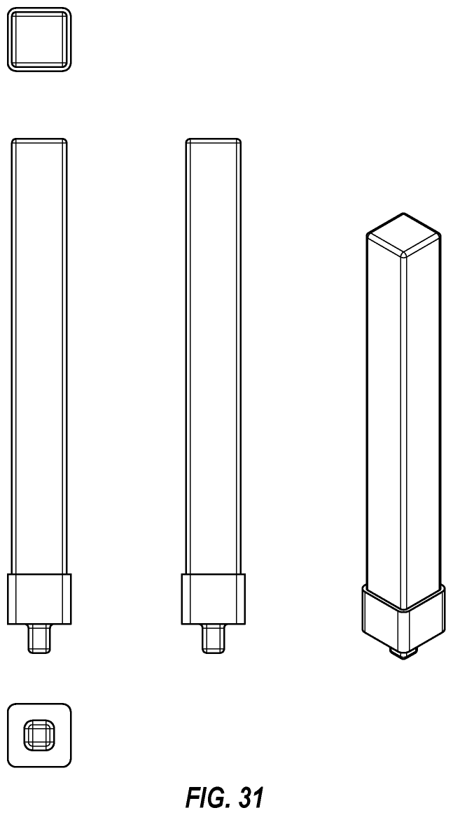

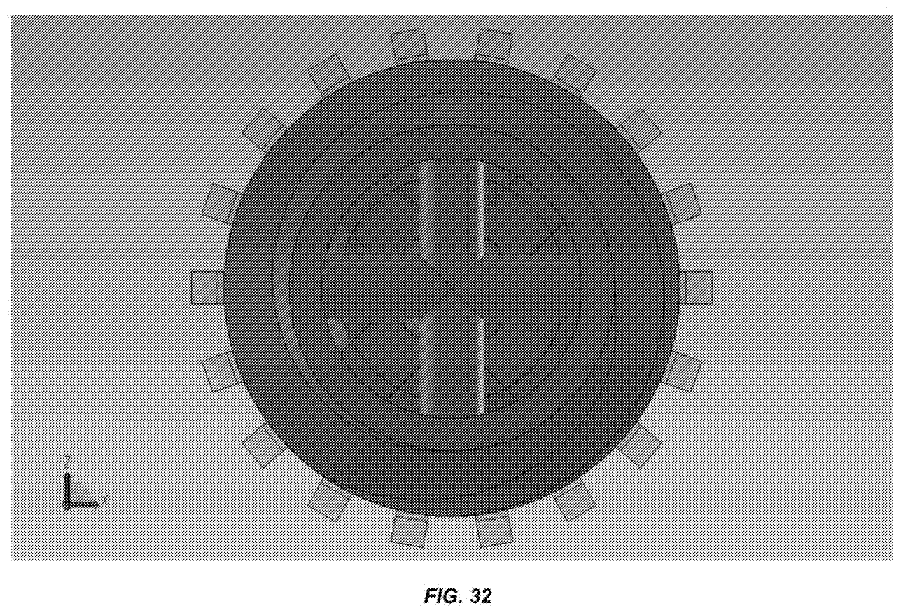

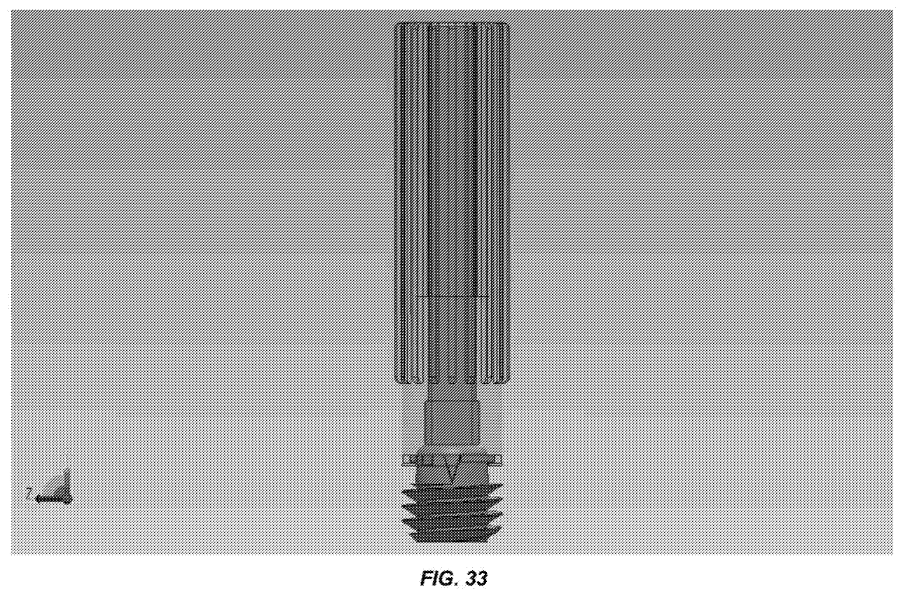

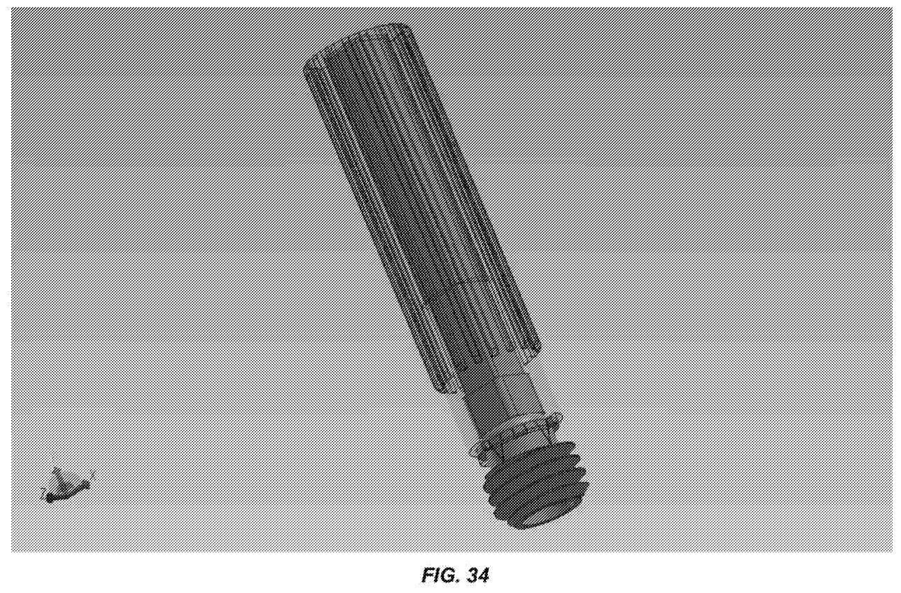

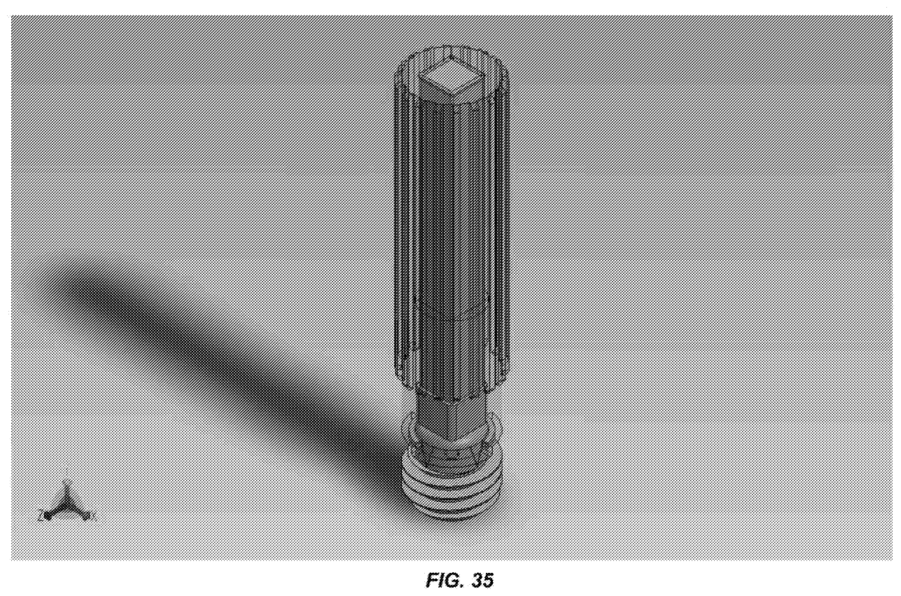

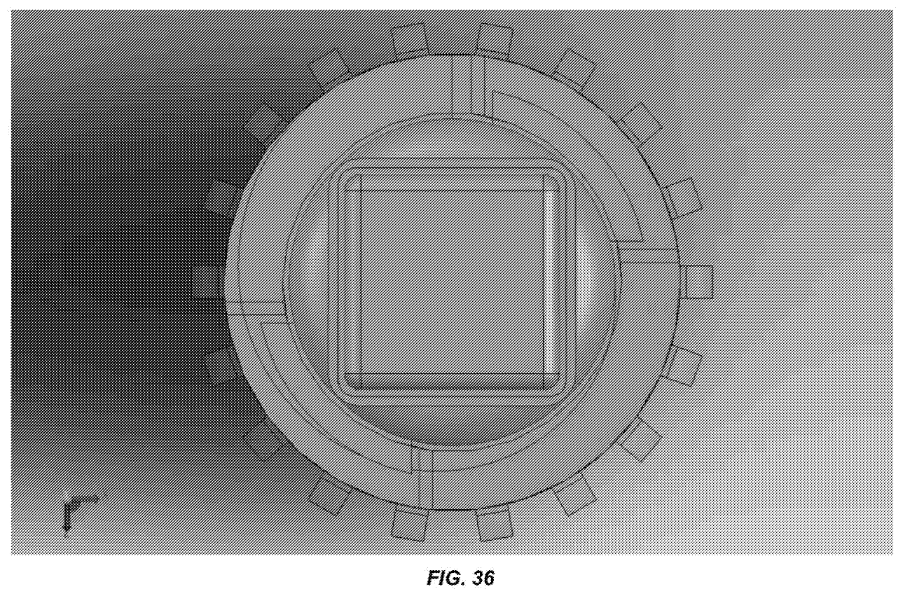

FIG. 4 provides a perspective view of the FIG. 3 cap. As shown in this view as a cube-shaped void, the alignment mechanism (i.e., aligning cube) can include an indentation or groove that interacts with a complementary alignment mechanism located on the applicator. Referring to FIG. 27 and FIG. 29, for example, the alignment mechanism can include an indentation or groove in the cap itself (i.e., without an aligning cube) that interacts with a complementary alignment mechanism located on the applicator.

As can also be seen in the FIG. 4 embodiment, the structure of the locking mechanism can extend laterally at least partially beyond the cap. When so designed, the extending portion of the locking mechanism can interact with the flanges of the anchor to further secure the cap within or on the anchor.

FIG. 5 shows a bottom perspective view of an applicator, under one embodiment. In this embodiment, the application comprises an applicator cylinder and a dispenser. The exemplary applicator of FIG. 5 comprises an alignment mechanism on the dispenser complementary to that on the aligning cube. As can be seen in the embodiment shown in FIG. 4 and FIG. 5, the complementary alignment mechanism is configured to fit within the cube-shaped alignment mechanism of the cap. In this embodiment, the dispenser is disposed within the applicator cylinder. The dispenser is configured to deploy the cap onto the anchor, which serves to lock the suture or soft tissue within the anchor. In certain embodiments, the applicator further includes at least one aligning bar that fits within the aligning notches of the anchor. Thus, in operation, the applicator serves to hold the cap, appropriately align the cap, and dispense the cap into the anchor.

As seen in FIG. 27, the applicator surface can comprise a textured surface, such as that for enhancing one's grip thereof. For example, the textured surface can be a non-slip surface. The applicator can be composed of a material that can be sterilized, such as by standard means. The ability to be sterilized is important, as embodiments of the applicator can be made from a re-usable material.

In embodiments, the applicator can be configured with a mating feature to allow the user to screw in the anchor prior to cap placement. In such embodiments, any shape, configuration, or design is appropriate, as long as the applicator and anchor are configured to interact with one another.

Although the alignment mechanism is shown as cube-shaped structure in the exemplary embodiments, any shape, configuration, or design is appropriate, as long as the alignment mechanism of the cap and the applicator are configured to interact with one another.

Under certain embodiments, the aligning notches are disposed on the applicator and the aligning bars are disposed on the anchor. Aligning notches and aligning bars can comprise any shape, size, or configuration.

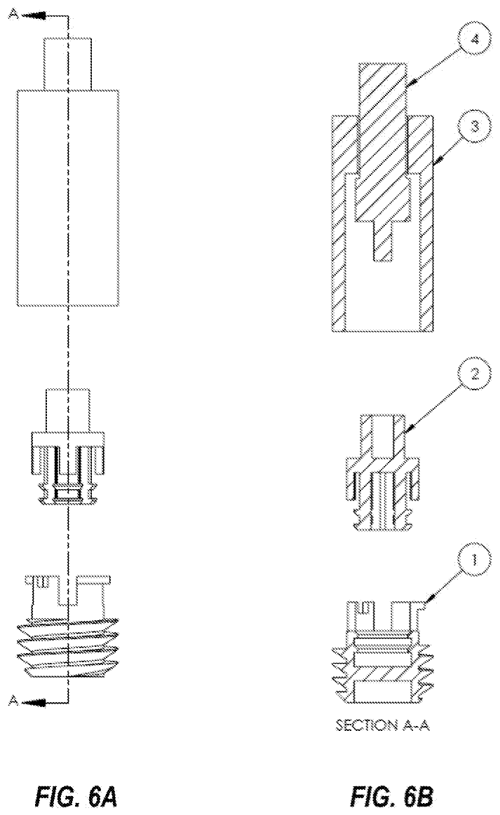

FIG. 6A shows a side schematic view of the surgical line fixation system, and FIG. 6B shows a cross sectional view of the surgical line fixation system, under an alternative aspect. The top of the figure show the applicator exploded above the cap, and the cap is shown exploded above the anchor. As can be seen in this embodiment, the locking mechanism of the cap comprises a structure that is substantially rectangular in shape. However, the skilled artisan will recognize that any shape of the locking mechanism can be utilized as long as the shape of the locking mechanism is complementary to the one or more locking mechanisms of the anchor. For example, the locking mechanisms can comprise flanges, wedges, notches, grooves, gaps, channels, or indentations of the anchor. As shown in the FIG. 6B embodiment, the applicator comprises a housing (3) that surrounds the dispenser (4). In this embodiment, the dispenser (4) comprises a handle that extends through the top of the housing, and the alignment mechanism can be seen as a rectangle-protrusion at the bottom of the dispenser. Above the protrusion, a widening of the dispenser is observed. This widening prevents the dispenser from moving up through applicator when in use.

The cap of the embodiment (2) shown in FIGS. 6A & 6B includes a securing mechanism that extends below the structure of the rectangular locking mechanism. The securing mechanism further includes a plurality of flanges. The plurality of flanges on the securing mechanism is configured to engage the flanges on the interior surface of the anchor, such that the cap is locked into place when affixed to the anchor.

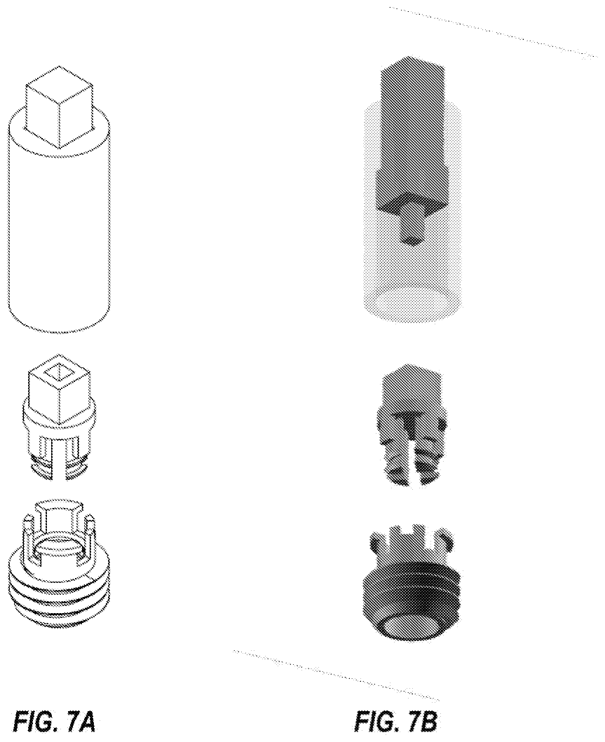

FIG. 7A shows a top perspective view of the exploded surgical line fixation system of FIG. 6A. Here, the cube-shaped void in the alignment mechanism of the cap is visible on the upper surface of the cap.

FIG. 7B provides a bottom perspective view of the exploded surgical line fixation system of FIG. 6A. In this view, the external housing of the applicator is shown in phantom to reveal the dispenser therein.

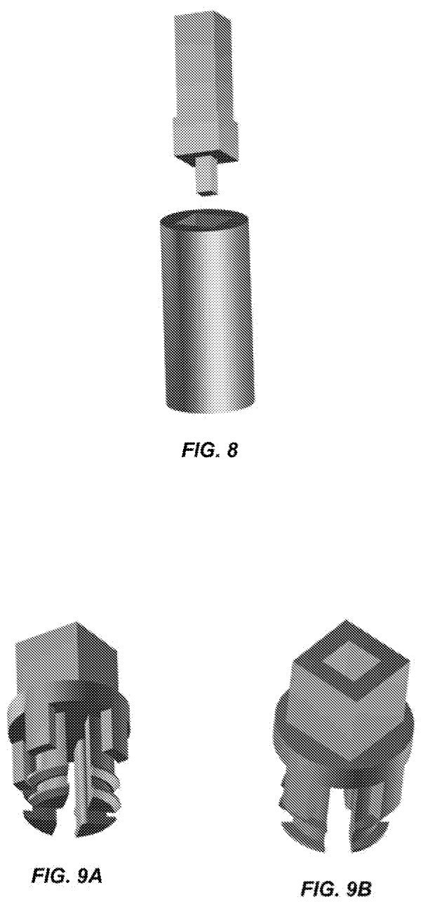

FIG. 8 provides a side perspective view of the applicator of FIG. 6A with the dispenser exploded above the external housing of the applicator. Here, a square-shaped void is visible in the external housing of the applicator through which the handle of the dispenser extends when disposed therein.

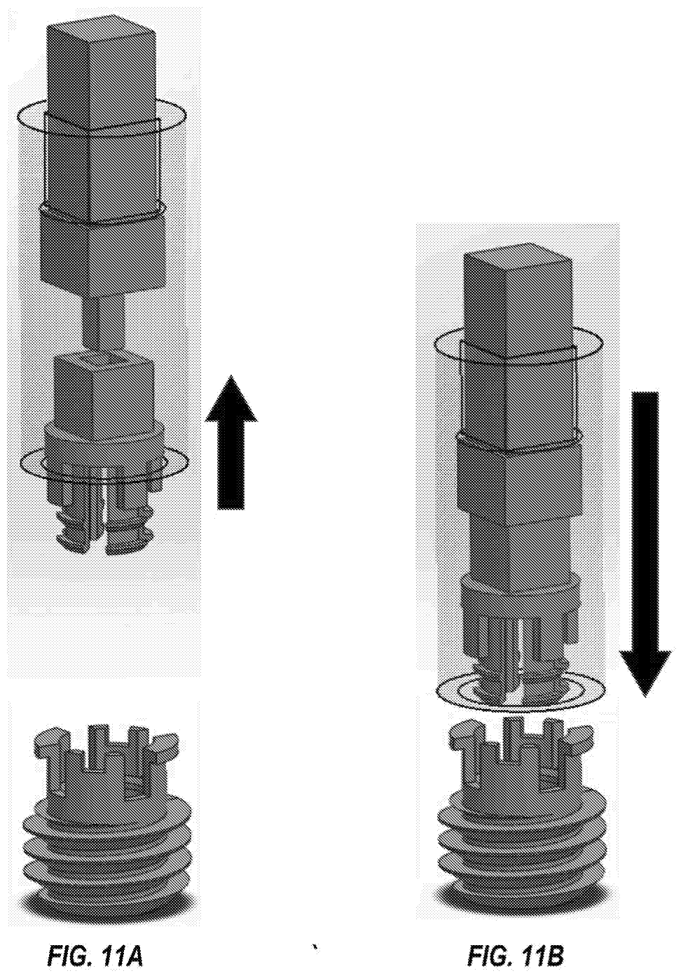

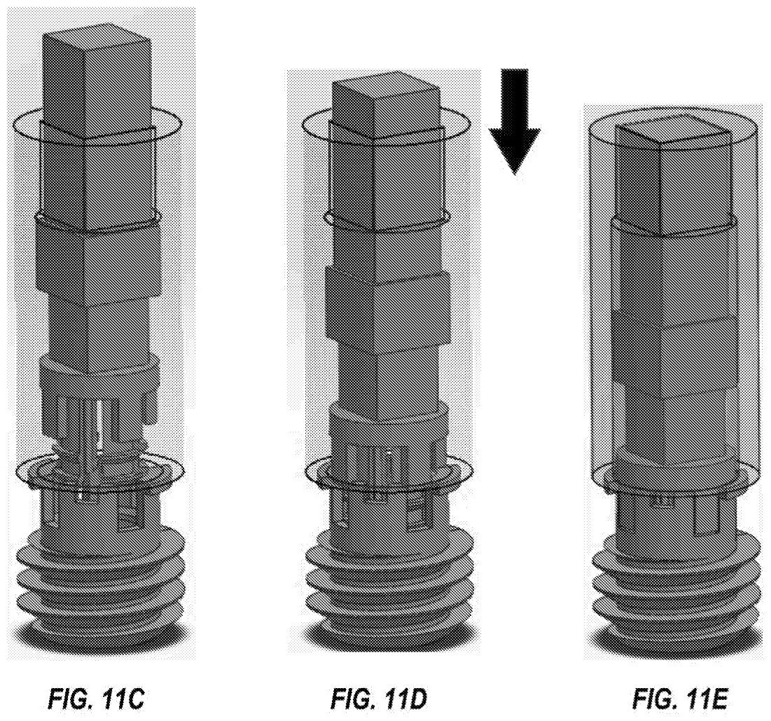

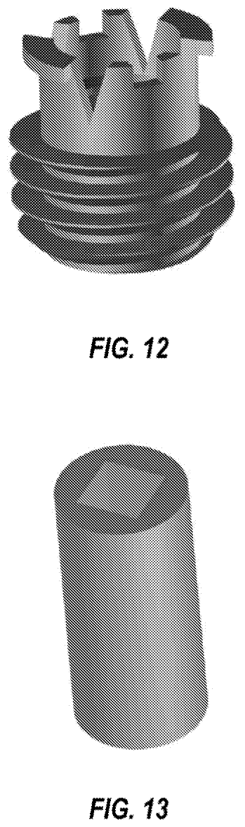

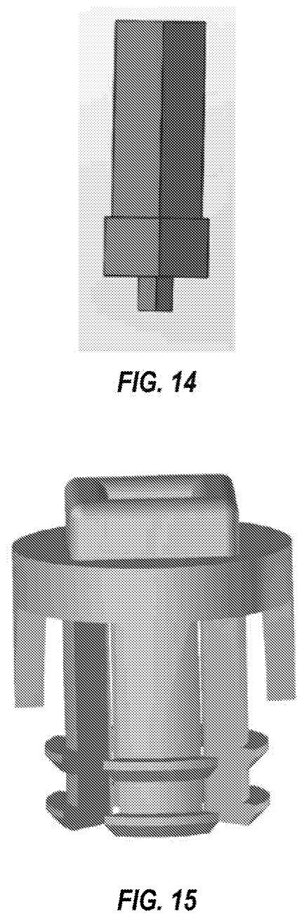

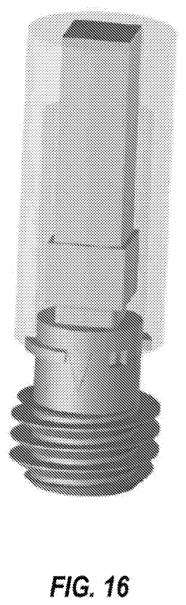

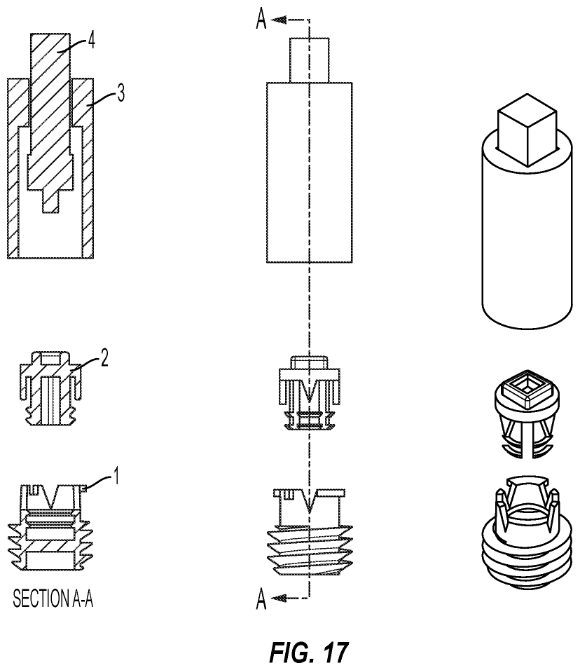

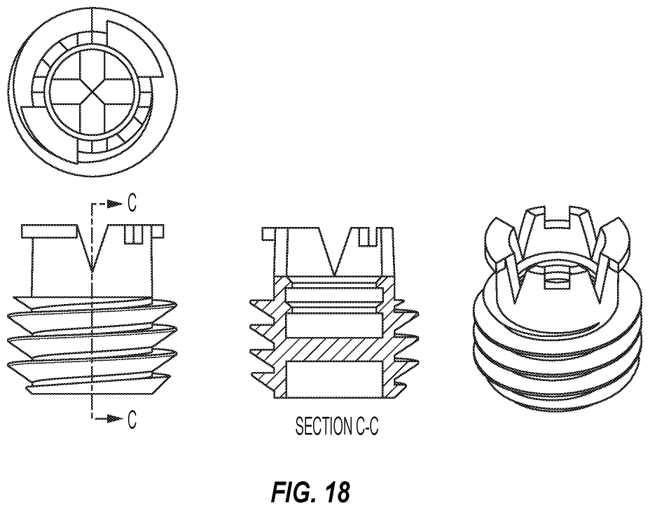

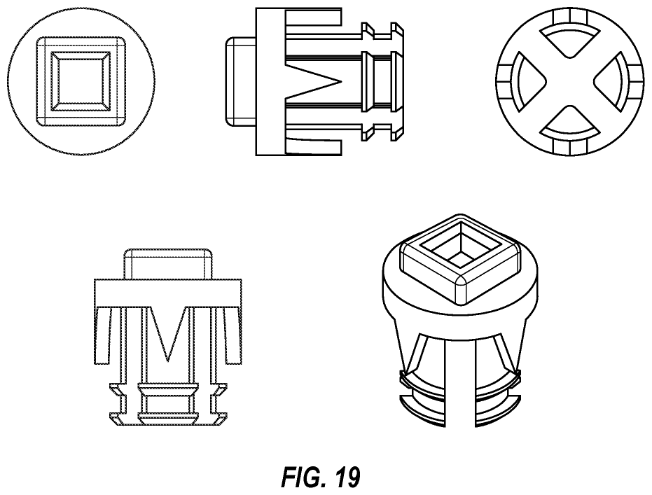

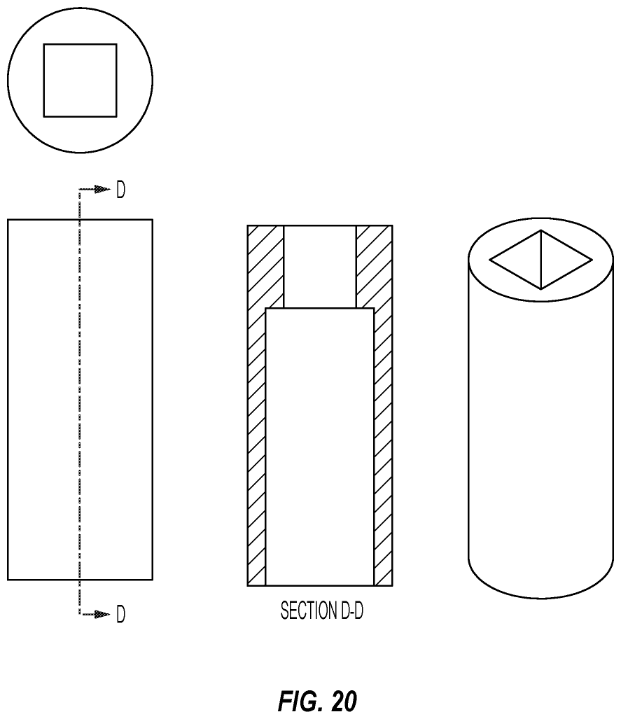

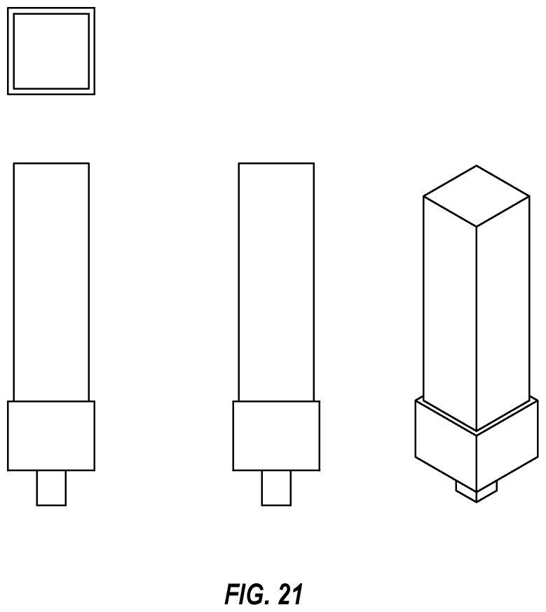

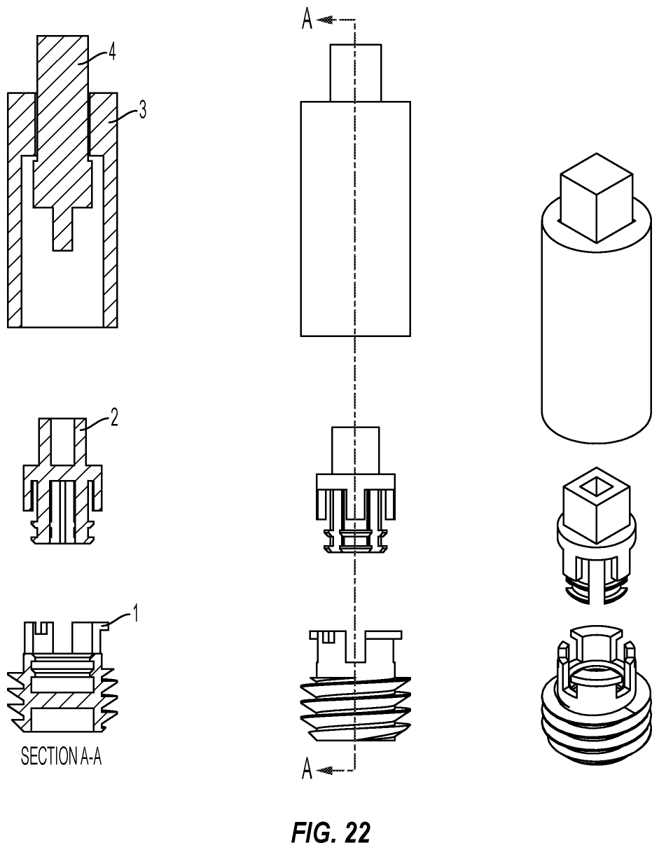

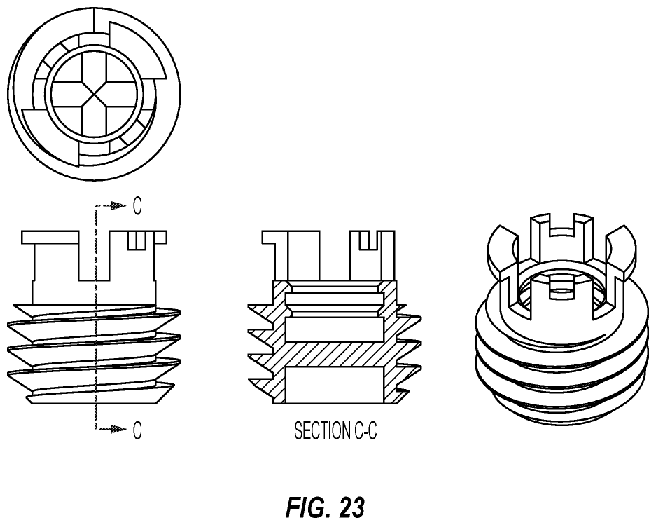

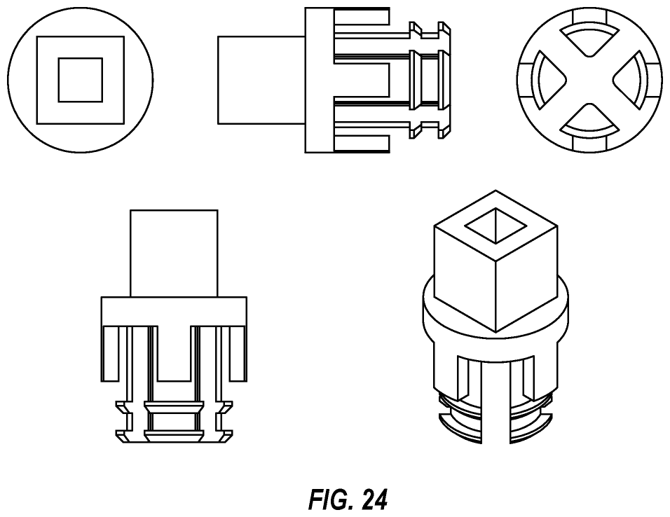

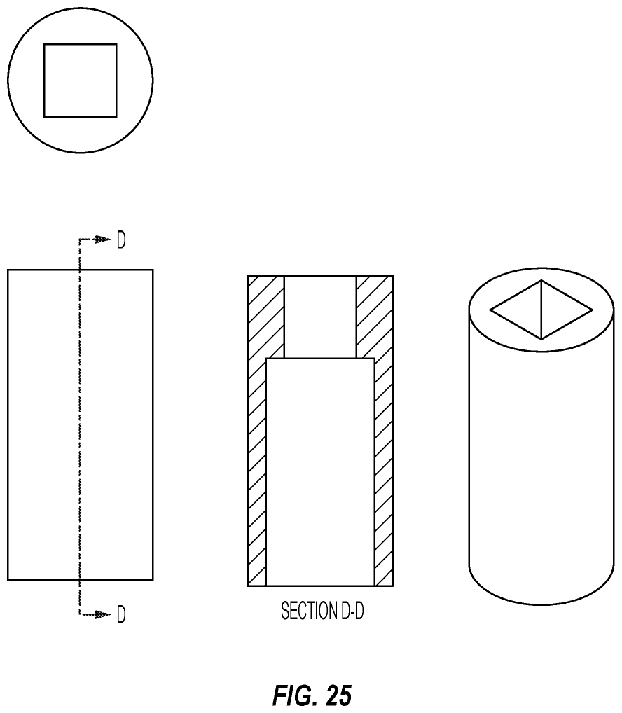

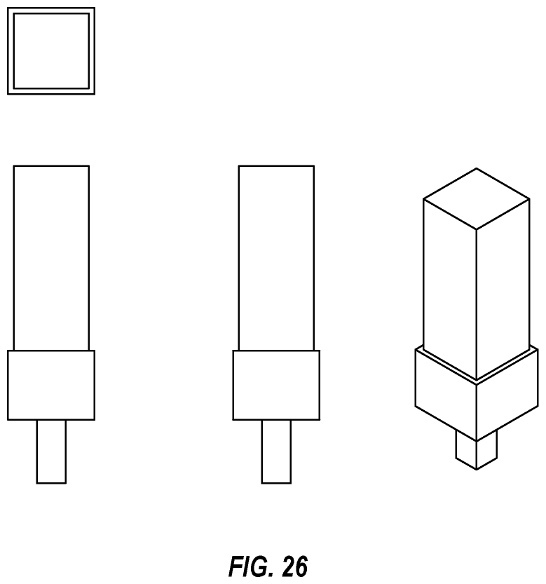

FIG. 9A provides a bottom perspective view of the cap of FIG. 6A. In this view, it is apparent that the securing mechanism of the cap comprises four structures that are integral with the cap and extend downwardly therefrom. The FIG. 9A view provides a clear picture of the flanges on the securing mechanism. In the top perspective view of the cap (FIG. 9B), the cube-shaped void of the alignment mechanism is visible. In operation, the alignment mechanism of the dispenser is inserted into the cube-shaped void before the cap is guided onto the anchor (discussed in more detail with regard to FIG. 11, below).

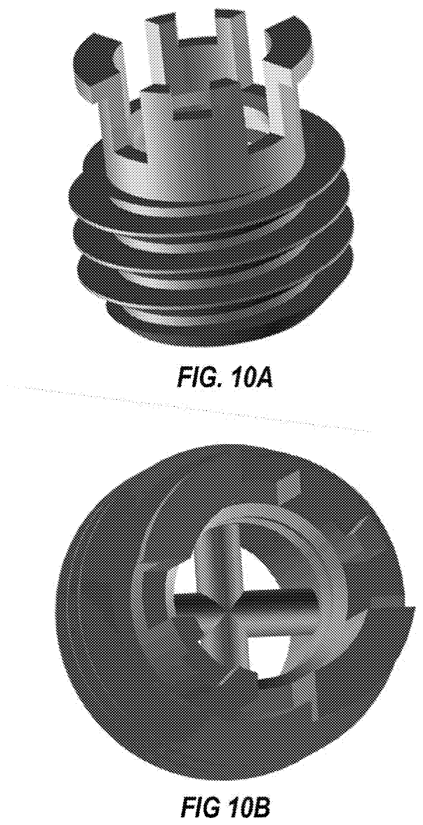

FIG. 10A shows a perspective view of the anchor of FIG. 6A. The bottom of the anchor comprises a series of threads configured to permit the anchor to reside within the hard tissue of a subject. As can be seen in this embodiment, the FIG. 10A anchor comprises four elongated notches, grooves, gaps, channels, or indentations that are configured to receive the locking mechanism of the FIG. 9 cap. FIG. 10B provides a top perspective view of the anchor under the FIG. 6A embodiment. Two crossbars can be seen traversing the interior space of the anchor, and the flanges are visible on the interior surface of the anchor.

When viewed in combination with the cap of FIG. 9, it is apparent that the four integral structures of the securing mechanism are configured to fit within each of the four the spaces between the cross bars of the FIG. 10 anchor. Thus, when placed over the anchor, the securing mechanism of the cap extends into the interior space of the anchor, and the flanges of the securing mechanism engage the flanges of the anchor to lock the cap in place.

FIGS. 11A-11E provide serial images of the exemplary mechanism by which the FIG. 6A surgical line fixation system works together. In all of FIGS. 11A-11E, the external housing of the applicator is shown in phantom. In FIG. 11A, the cap is moved in the direction of the arrow. As shown, the cube-shaped void of the alignment mechanism of the cap is disposed directly beneath the protruding alignment mechanism of the dispenser. Thus, as the cap moves upward, in the direction of the arrow, the cube-shaped void of the cap receives the alignment mechanism of the dispenser, and the applicator and the cap form an applicator/cap complex.

FIG. 11B shows the applicator/cap complex moving in the direction of the arrow, toward the anchor. As shown in this figure, the cap is contained within the external housing of the applicator.

In FIG. 11C, the bottom surface of the external housing of the applicator comes into contact with the upper surface of the anchor, and the applicator/cap complex is aligned for deployment of the cap.

In FIG. 11D, the handle of the dispenser is depressed in the direction of the arrow. As the handle is depressed, the dispenser lowers the cap into position over and within the anchor.

FIG. 11E shows the dispenser of the applicator fully depressed, and the cap is now affixed to the anchor to form a cap/anchor complex. In operation, the applicator housing and the dispenser are then removed from the cap/anchor complex. In certain embodiments, the alignment mechanism of the cap is also removed such that the top of the cap is flush with the surface of the rigid structure or hard tissue of the subject.

In alternative embodiments, the anchor fixation system is provided without an applicator. In such embodiments, the cap is aligned and secured to the anchor by hand.

The suture anchoring system can comprise any size, shape, or configuration appropriate for securing soft tissue to hard tissue. For example, such procedures can include rotator cuff repair, tendon and ligament avulsion, digital dorsal extensor mechanism repair, forehead lifting, face lifting, facial soft tissue suspension, suspension of abdominal and thoracic musculature to bone, suspension of temporalis muscle to bone, facal ligamentous suspension, facial feminization surgery, canthoplasty, canthopexy, midface lifting, and the like. The anchoring system can comprises a rounded, rather than a linear shape. In embodiments, the anchoring system disclosed herein comprises rounded edges. In embodiments, the anchor, cap, applicator, or a combination thereof comprises a rigid, non-flexible structure.

In embodiments, the anchor and cap of the presently disclosed system are configured to reside within the rigid structure. In certain embodiments, the top of the cap is flush with the surface of the rigid structure following installation of the anchoring system. Embodiments may reside underneath the surface of the rigid structure after installation.

Certain embodiments of the suture fixation system comprise a simple structure. Embodiments lack an eyelet or other mechanism through which a suture must be passed in order to achieve anchoring of the soft tissue. The term “soft tissue” refers to soft materials from living organisms. The suture fixation system can be configured to work with or without requiring suture knots. In embodiments, the suture fixation system does not require knots within the suture line.

The diameter of the cap can be up to 50 mm. The cap can comprise a diameter of up to 10 mm. In an embodiment, the cap comprises a diameter of about 4 mm. The wedges of the cap can be about 3 mm wide at the base and about 0.5 mm wide at the apex. The aligning mechanism can be any of various sizes and shapes. In embodiments with a cube-shaped alignment mechanism, the aligning cube of the cap can comprise walls that are about 2 mm-long. The height of the aligning cube can be about 5 mm.

The diameter of the applicator can be up to 60 mm. The applicator can comprise a diameter of up to 15 mm. In one embodiment, the outer diameter of the applicator is about 6.5 mm. The outer diameter can be between 0.5 to 6.5 mm, inclusive. The interior diameter off the applicator can be about 4.5 mm. In embodiments, the height of the applicator can be up to 1000 mm. The applicator height can be up to 500 mm. In one embodiment, the applicator is about 400 mmm high. The applicator can be between 100 and 400 mm in height, inclusive. The applicator can comprise a height of less than 400 mm. The dispenser (or plunger) can comprise a diameter of up to 30 mm and a height of up to 2,500 mm. The diameter of the dispenser can be up to 20 mm and the height can be up to 1000 mm. In one embodiment, the diameter of the dispenser is about 6 mm, and the height is about 500 mm. The dispenser diameter can be less than 6 mm, and the height can be less than 500 mm.

As many as 50 sutures may be affixed to the suture anchor. In embodiments, up to 20 sutures can be affixed to a single suture anchor. Certain embodiments can affix up to 10 sutures. In embodiments, one, two, three, four, five, six, seven, eight, nine, or ten sutures can be affixed to the anchor.

One of ordinary skill in the art will readily appreciate that the dimensions of the various components of the suture anchoring system will vary depending upon the desired surgical applications. For example, smaller dimensions can be useful for periorbital and digital (phalangeal) surgeries, whereas larger dimensions can be useful for rotator cuff and tendon/ligament avulsion repairs.

The skilled artisan will recognize that components of the suture anchoring system can be composed of various materials dependent upon the desired physical and mechanical properties, such as those described herein.

For example, the material can be a biocompatible material. The term “biocompatible” can refer to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

For example, the material can be medical grade stainless steel, titanium alloy, polylactic/polyglycolic acid, polyether ether ketone (PEEK), methylmethacrylate, polydioxanone, and the like. The substrate can be composed of about 10%, 20%, 30%, 40%, 50%, 60%, 70%, 80%, 90%, or 100% of a biocompatible material.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The material can be a bioabsorbable material, including synthetic bioabsorbable materials or natural bioabsorbable materials. Non-limiting examples of such materials include polylactic acid, polyglycolic acid, polycaprolactone, or combinations thereof. A large number of different types of materials are known in the art which may be inserted within the body and later dissipate. The term “bioabsorbable” is used herein to refer to materials selected to dissipate upon implantation within a body, independent of which mechanisms by which dissipation can occur, such as dissolution, degradation, absorption and excretion. The terms “bioabsorbable,” “resorbable” or “biodegradable” are used synonymously herein, unless otherwise specified, to refer to the ability of the material or its degradation products to be removed by biological events, such as by fluid transport away from the site of implantation or by cellular activity (e.g., phagocytosis). Only the term “bioabsorbable” will be used in the following description to encompass absorbable, bioabsorbable, and biodegradable, without implying the exclusion of the other classes of materials.

The material can be a polymeric material, such as any of various plastics or combinations of plastics. “Non-bioabsorbable” material refers to a material, such as a polymer or copolymer, which remains in the body without substantial bioabsorption. Non-limiting examples of non-bioabsorbable materials comprise metals or plastic.

The suture anchor can be molded, casted, sculpted, 3-D printed, or otherwise created.

Methods of Use

The development of suture anchoring devices has played an important role in the transition of from open to arthroscopic surgical procedures, and they are currently a growing component of the consumables segment of the market for orthopedic and arthroscopic surgery. The commercial market for suture anchor devices is forecast to be driven by an increasing geriatric population, which is resulting in an increase in the prevalence of musculoskeletal disorders, especially osteoarthritis and rheumatoid arthritis; an increasing incidence of sports injuries; the cost effectiveness of arthroscopic surgeries compared to open surgeries; technological advancement in arthroscopic implants and suture anchor devices; and the rising adoption of minimally invasive surgeries.

Thus, aspects of the invention further provide for methods of using the suture anchoring device described herein for these and other applications. Embodiments are suitable for use in any rigid structure or hard tissue of a subject and are capable of being inserted anywhere within or on a subject's body. For example, embodiments herein can be used for surgical procedures such as anterior cruciate ligament reconstruction; rotator cuff repair; plastic/reconstructive face and head soft tissue surgery; elbow/angle tendon and ligament repair/stabilization; cleft palate, temporal mandibular joint ligaments and articular disc repair. Additional surgical procedures include facial muscle anchoring, facial paralysis reconstruction, cosmetic facial surgical procedures, including browlift and canthoplasty, rhytidectomy, platysmaplasty; hand tendon repair; belt lipectomy; thigh lifting; upper extremity tendon and muscle reconstruction; temporalis muscle suspension; medial canthal reconstruction; facial soft tissue suspension; or combinations thereof.

In embodiments, the suture anchoring device and/or components thereof are implanted within a subject. As used herein, the term “subject” can refer to any mammal, including but not limited to human beings, pigs, rats, mice, dogs, cats, goats, sheep, horses, monkeys, apes, rabbits, and cattle. The terms “subject” and “patient” can be used interchangeably.

As used herein, the term “implanted” can refer to an ability of a medical device such as the anchor and, optionally, the cap, to be positioned at a location within a body, such as a body vessel. Furthermore, the terms “implantation” and “implanted” refer to the positioning of a medical device at a location within a body of a subject.

The presently disclosed methods permit adjustable tensioning of the sutures or soft tissue before fixing the sutures or soft tissue within the anchor. In one embodiment, a hole is drilled into hard tissue of a subject. The term “hard tissue” can refer to hard materials from living organisms, non-limiting examples of which comprise bone, cartilage, teeth, nails, hair, and the like. The anchor can be inserted into the hole. In embodiments, the anchor is advanced into place by force and/or by rotation. For example, rotation can effectively screw the anchor into place in a bone or tissue. In other embodiments, force can be used to push the anchor in place such that flanges expand on the other side of a bony shelf. The sutures are threaded around the crossbars of the anchor. Next, the suture is passed through soft tissue of a subject, and the suture is temporarily secured within the notch of the suture anchor for stability. This process is repeated until the required number of sutures is passed through the soft tissue. Once all desired sutures are passed through tissue and reversibly engaged in the anchor, the tension of the sutures can be adjusted by freeing the suture from the notch, increasing or decreasing tension and then reengaging the suture in the notch. After the desired level of tension is achieved, the sutures are affixed in place by securing the cap onto the anchor. In certain embodiments, the sutures are tied in place within the anchor before the cap is affixed thereon.

In embodiments, the cap is affixed to the anchor as generally shown in FIGS. 11A-11E and described in detail above.

In an embodiment, the cap is retained within the column of the application plunger while it is used to apply and stabilize the base. In this embodiment, the rod within the plunger will be removable and only applied from the top to apply the cap. This will allow the surgeon to place the palm of the hand over the top of the column of the application plunger when placing an anchor. Further, there will be depressions on opposite sides of the plunger column to allow for fingers to grip the column while the palm of the hand applies pressuring during application of the anchor.

One or more knots can be tied into the sutures to prevent the sutures from accidentally being pulled through the anchor or tissue. The methods can employ sutures with a single needle or a needle at either end of the suture.

To achieve enhanced stability, the sutures can be engaged in two notches once the desired tension is achieved. In such embodiments, the suture is engaged in the apex of at least two notches.

For embodiments without sutures, the process will be the same, but soft tissue will be engaged directly in the notch rather than sutures. Embodiments can use a combination of sutures and tissue sections to secure the soft tissue to the hard tissue.

In embodiments that comprise an applicator, the alignment mechanism of the applicator is joined with the alignment mechanism of the cap. The aligning bars of the applicator can then be placed within the aligning notches of the anchor to ensure proper orientation of the cap. When properly aligned, the dispenser is engaged such that the cap is secured onto the anchor. When so secured, the sutures or tissue are secured into the anchor, thereby affixing the soft tissue to the hard tissue.

The procedure for installing the suture anchor of the present invention is similar to the procedure many surgeons are familiar with for installing existing suture anchors. That is, the area for anchor placement is surgically isolated with blunt and sharp dissection, a tunnel is created by drilling, the hole is tapped (in the case of a threaded anchor with non-cutting threads), or not, the anchor is placed, the sutures passed through tissues, tension adjusted, and the cap placed.

Kits

Further, aspects of the invention provide for kits comprising the suture anchoring device described herein and instructions for use. For example, the kit can comprise one or more of the following components: anchor; cap and aligning tube; sutures, such as preplaced sutures; applicator which affixes cap to anchor. For instance, the kit can comprise components useful for carrying out methods of the invention and instructional material that describes the method of utilizing the suture anchoring system as described herein.

In certain embodiments, the kits can comprise anchors of various sizes, wherein the appropriate size is selected according to the needs of the patient, the preference of the doctor, or a combination thereof. Kit embodiments can further include a drill.

In certain embodiments, the kit comprises instructional material.

Instructional material may include a publication, a recording, a diagram, or any other medium of expression which can be used to communicate the usefulness of the device or kit described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet. The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Sutures are pre-threaded around the crossbars at the interface of the threaded and unthreaded portions of the device. The threaded portion is advanced into a bone tunnel. A loop of suture with a knot rests just outside the deepest portion of the “V” shaped notches. Each suture is looped around one of the crossbars inside the hollow device. The sutures are passed through soft tissue and back through the loop outside of the device. It is temporarily secured in the apex of the notch next to the other strand of and the notch directly opposite. Each suture is independently passed through the tissue and temporarily secured similarly. Once all sutures are tensioned to the surgeon's preference, a security cap is applied to permanently fix the sutures in place with the cap applicator. There are flanges on the inside of the device that secure the cap in the hollow device surface. It cannot be removed once in place. If it is necessary to remove the device, the sutures are cut and the device backed out of the bone.

The suture anchoring device permits numerous suture anchors, for example 2-10, in a single device. Each suture is thus optimally and independently tensioned prior to securing all sutures by lowering the securing disc into the top portion of the device. The suture anchoring device can be used for anterior cruciate ligament reconstruction, rotator cuff repair, plastic/reconstructive face and head soft tissue surgery, brow lifts, temporalis muscle re-suspension, facial paralysis correction, hand ligamentous and tendon reconstruction, elbow/angle tendon and ligament repair/stabilization, and for cleft palate/temporal mandibular joint ligaments and articular disc repair.

Advantages of embodiments described herein over existing devices currently available include, for example, multiple sutures for only 1 anchor; independent control of suture tension; no knots or tying is required; can be optimized/designed for multiple uses with minimum revision with applications ranging from plastic surgery to ACL repair; easily printed/molded; no changes in current procedures are needed; embodiments can comprise non-absorbable, absorbable, and biocomposite formulations; and should the anchor be unstable in the local bone environment, another slightly larger size will be available in the kit.

This device overcomes current limitations of traditional bone anchors and will significantly advance mechanisms of securing soft tissue to bone.

### Example 2

HoldTen Suture Ancho Manufacturing and Clinical Development

Partial or full detachment from the bone of ligaments, tendons and other tissues are common knee and shoulder injuries. While these tissues can re-attach without intervention, in some cases, especially with complete detachment, surgery may be necessary. An arthroscope is a medical device that is commonly used in such surgeries to visualize, examine, and execute therapeutic interventions inside the joints of the body, such as knee, hip, spine, shoulder, and elbow in conditions, such as osteoarthritis, rheumatoid arthritis, tendinitis, and bone tumor. Here, suture anchors are used to help attach soft tissue to bone facilitate healing.

The development of suture anchoring devices has played an important role in the transition of from open to arthroscopic surgical procedures, and they are currently a growing component of the consumables segment of the market for orthopedic and arthroscopic surgery. Bio-composite suture anchors dominate the global market and PEEK suture anchors are the second largest segment.

Despite a growing clinical demand, there have been reported complications during use of suture anchors and suture anchor devices, including loosening, migration, and chondral injury. Embodiments as described herein provide a novel surgical line fixation tool, HoldTen, which has design improvements on current suture anchor devices that greatly position it to make it clinically and commercially impactful technology. HoldTen can be used in various surgical applications ranging from orthopedics, dentistry, upper airway repair, and craniofacial plastic and reconstructive surgery. Additionally, its design allows for modifications that permit use in different surgical specialties.

The device offers the competitive advantages of the ability to attach multiple sutures to a single device and the ability to adjust the tension of sutures. Furthermore, market dynamics suggest that that it will be important to be able to make the product with biodegradable materials.

Current surgical suture anchoring devices utilize deployable anchoring devices in which the anchor is physically deployed after the device is bored into cartilaginous tissues or bones. Once deployed, the anchor cannot be removed and the fixation is irreversible. The inherent design of current suture anchors prevents the removal of sutures as they are fixed to the core component of the device.

Moreover, the design limits the device to one suture per device in which the tension cannot be adjusted due to the knots required to secure the tissue. Such limitations complicate the suturing process and extend time spent in surgery. Reported patient complications exist such as anchor loosening, migration, or chondral injury. Consequently, these patient complications may precipitate the need for additional surgeries costing both the surgeon and patient valuable time.

The HoldTen is a surgical line fixation device that overcomes challenges and limitations of available surgical suture anchoring devices. Key features of the device include:
Multiple sutures per anchor
Ability to adjust tension of sutures on anchor
Also possible to secure soft tissue to bone
Provided as a kit with the following components: a) Anchor b) Cap and Aligning Tube c) Preplaced sutures d) Applicator-affixes cap to anchor

The market for resorbable and titanium hardware for surgical applications is large, growing, and carries high margins. Based on our clinical experience with suture anchors, screws, and plates from four commercial plate and screw manufacturers, we will develop adequate prototypes to hold large-diameter sutures, and small diameter sutures for use in orthopedic knee reconstruction, and plastic surgery craniofacial applications, respectively. Once developed and manufactured, testing in human cadaver models can determine the clinical utility of the product. This would then be followed by limited trials in human clinical cases.

We will first develop HoldTen prototypes in virtual space using STL software and our knowledge of human bone depth, soft tissue thicknesses throughout the body, and comparative screw sizes available clinically. Next, we will manufacture titanium and polylactic/polyglycolic acid prototypes for craniofacial and knee procedures. Then we will iteratively test these prototypes in human cadavers for refinement before finally conducting market evaluation of manufacturing supply chains and points of sale to determine licensing vs independent firm creation for market entry.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A surgical line fixation device comprising:
an anchor configured to at least partially reside within a rigid structure, the anchor being further configured to hold one or more sutures; and
a cap configured to secure the one or more sutures within the anchor.

2. The surgical line fixation device of claim 1, wherein the anchor comprises a top portion and a bottom portion, wherein the top portion is configured to hold one or more sutures, wherein the top portion is configured to receive the cap, and wherein the bottom portion is configured to permit attachment of the anchor to the rigid structure.

3. The device of claim 1, wherein the anchor comprises an interior space with one or more crossbars disposed therein;
the one or more crossbars being configured to receive and hold at least a portion of a suture.

4. The device of claim 1, wherein the anchor comprises one or more sections configured to receive and hold at least a portion of the suture.

5. The device of claim 4, wherein the one or more sections comprise wedges, notches, grooves, gaps, channels, or indentions.

6. The device of claim 2, wherein the top portion comprises one or more locking mechanisms, wherein the one or more locking mechanisms comprise a structure that is complementary to one or more locking mechanisms on the cap.

7. The device of claim 1, wherein the cap comprises one or more locking mechanisms, wherein the one or more locking mechanisms comprise a structure that is complementary to one or more locking mechanisms on the anchor.

8. The device of claim 1, wherein the cap comprises one or more securing mechanisms, wherein the one or more securing mechanisms comprise a structure that is complementary to one or more securing mechanisms on the anchor.

9. The device of claim 2, wherein the top portion further comprises one or more securing mechanisms, wherein the securing mechanisms are configured to receive and secure the anchor to a cap.

10. The device of claim 1, wherein the anchor comprises a threaded portion configured to permit attachment of the anchor to the rigid structure.

11. The device of claim 1, wherein the anchor comprises a flange configured to permit attachment of the anchor to the rigid structure.

12. The device of any one of claims 1-11, further comprising an applicator, the applicator comprising a dispenser and a first alignment mechanism, wherein the first alignment mechanism is configured to interact with a second alignment mechanism disposed upon the cap.

13. The device of claim 12, wherein the first and second alignment mechanisms are complementary to one another.

14. The device of claim 12, wherein the second alignment mechanism is configured to receive the first alignment mechanism.

15. The device of claim 12, wherein the first alignment mechanism is configured to receive the second alignment mechanism.

16. The device of claim 12, wherein the second alignment mechanism is removable.

17. The device of claim 12, wherein the applicator further comprises one or more aligning bars and the anchor further comprises one or more aligning notches configured to receive the aligning bars.

18. The device of claim 17, wherein the aligning notches configured to receive the aligning bars are located on the top portion of the anchor.

19. The device of claim 10, wherein the anchor further comprises one or more aligning bars and the applicator further comprises one or more aligning notches configured to receive the aligning bars.

20. The device of claim 19, wherein the aligning bars configures to receive the aligning notches are located on the top portion of the anchor.

21. The device of claim 1, wherein the rigid structure comprises a hard tissue of a subject.

22. The device of claim 1, wherein the rigid structure comprises cartilaginous tissue, a bone, or a tooth of the subject.

23. The device of claim 1, wherein the rigid structure comprises cortical bone, cancellous bone, flat bones, long bones, enamel, cartilage, or fibrocartilage.

24. The device of claim 1, wherein the device is substantially cylindrical.

25. The device of claim 1, wherein the device is polygonal.

26. The device of claim 1, wherein the anchor is configured to be removable when least partially residing within the rigid structure.

27. The device of claim 1, wherein the anchor is configured to reversibly hold the one or more sutures.

28. The device of claim 1, wherein the anchor is configured to be permanent or semi-permanent.

29. The device of claim 1, wherein the device is configured to hold up to 20 sutures.

30. The device of claim 1, wherein the device is configured to hold up to 10 sutures.

31. The device of claim 1, wherein the device is configured to hold up to 5 sutures.

32. The device of claim 1, wherein the device is configured to permit adjustment of a tension within each of the one or more sutures.

33. The device of claim 3, wherein the interior space defines an interior surface of the anchor, and the interior surface comprises one or more flanges configured to secure the cap to the anchor.

34. The device of claim 3, wherein the interior space defines an interior surface;
the interior surface comprises a first plurality of threads;
the cap comprises a second plurality of threads; and
the first plurality of threads and the second plurality of threads are configured to engage one another.

35. The device of claim 1, wherein the device comprises a metal or a synthetic polymer.

36. The device of claim 1, wherein the device comprises a bioprinted material.

37. The device of claim 36, wherein the bioprinted material comprises a natural or synthetic protein, a mineral, or a combination thereof.

38. The device of claim 37, wherein the protein comprises collagen, laminin, or a combination thereof.

39. The device of claim 37, wherein the mineral comprises tricalcium phosphate.

40. The device of claim 1 configured for use in anterior cruciate ligament reconstruction or repair, rotator cuff repair, plastic or reconstructive face or head soft tissue surgery, elbow or ankle tendon and ligament repair or stabilization, cleft palate repair, temporal mandibular joint ligaments or articular disc repair, dental applications, gum stabilization, upper airway surgery, facial muscle anchoring, facial paralysis reconstruction, cosmetic facial surgical procedures (including browlift and canthoplasty), rhytidectomy, platysmaplasty, hand tendon repair, belt lipectomy, thigh lifting, upper extremity tendon and muscle reconstruction, temporalis muscle suspension, medial canthal reconstruction, facial soft tissue suspension or combinations thereof.

41. The device of claim 40, wherein upper airway surgery comprises larynx repair, pharynx repair, or a combination thereof.

42. A method of securing soft tissue to a rigid structure comprising:
drilling a channel into the rigid structure;
inserting the anchor of the device of claim 1 into the rigid structure;
suturing a soft tissue to the anchor with the one or more sutures;
modifying the tension of the one or more sutures; and
placing the cap on the anchor to secure the sutures in place.

43. The method of claim 42, further comprising:
looping each of the one or more sutures around one or more crossbars disposed within an interior space of the anchor;
passing the one or more sutures through the soft tissue;
temporarily securing at least a portion of the one or more sutures within a section;
tensioning the one or more sutures; and
affixing the cap to the anchor.

44. The method of claim 43, wherein the cap further comprises one or more locking mechanisms;
the method further comprising aligning the one or more locking mechanisms with the one or more locking mechanisms of on the anchor before affixing the cap to the anchor.

45. The method of claim 42, 43, or 44, further comprising the applicator of claim 8, and further comprising the steps of:
aligning the first alignment mechanism with the second alignment mechanism; and
using the applicator to affix the cap to the anchor.

46. A kit for securing soft tissue to a rigid structure comprising the device of claim 1 and instructions for use.

47. A kit for securing soft tissue to a rigid structure comprising the device of claim 10 and instructions for use.

48. The kit of claim 46 or claim 47 further comprising a drill.

49. The kit of claim 46 or claim 47, wherein the anchor comprises a first threaded portion configured to permit reversible attachment of the anchor to the rigid structure; and
the kit further comprises a second anchor comprising a second threaded portion that is larger than the first threaded portion.

50. The kit of claim 46 or claim 47 further comprising a plurality of differentially sized anchors.

51. A kit for securing soft tissue to a rigid structure comprising:
a drill;
a plurality of differentially sized anchors, each anchor being configured to at least partially reside within a rigid structure, each anchor being further configured to hold one or more sutures;
a cap configured to secure the one or more sutures within the anchors;
an applicator comprising a dispenser and a first alignment mechanism, wherein the first alignment mechanism is configured to interact with a second alignment mechanism disposed upon the cap; or a combination thereof, and
instructions for use.

52. A surgical fixation device comprising:
an anchor configured to at least partially reside within a rigid structure, the anchor being further configured to hold one or more soft tissue sections; and
a cap configured to secure the one or soft tissue sections within the anchor.

53. The device of claim 52, wherein the anchor further comprises one or more sections configured to receive and hold at least a portion of the tissue sections; and
wherein the cap comprises one or more locking mechanisms, wherein the locking mechanisms each comprise a structure that is complementary to a locking mechanism on the anchor.

54. The device of claim 53, further comprising an applicator, the applicator comprising a dispenser and a first alignment mechanism, wherein the first alignment mechanism is configured to interact with a second alignment mechanism disposed upon the cap.

55. A method of securing soft tissue to a rigid structure comprising:
drilling a channel into the rigid structure;
inserting the anchor of the device of claim 51 into the rigid structure;
inserting one or more soft tissue sections into at least a portion of the anchor;
modifying the tension of the one or more soft tissue sections; and
placing the cap on the anchor to secure the one or more soft tissue sections in place.

56. The method of claim 55, further comprising:
temporarily securing at least a portion of the one or more soft tissue sections within a notch, groove, gap, channel, or indentation;
tensioning the one or more soft tissue sections; and
affixing the cap to the anchor.
